(19) United States
(12) Patent Application Publication
(10) Pub. No.: US 2023/0281417 A1
(43) Pub. Date: Sep. 7, 2023

(54) SMART CARD

(71) Applicant: Mileke Armein Davis, Lithonia, GA (US)

(72) Inventor: Mileke Armein Davis, Lithonia, GA (US)

(21) Appl. No.: 18/197,714

(22) Filed: May 15, 2023

Related U.S. Application Data
(63) Continuation-in-part of application No. 16/527,623, filed on Jul. 31, 2019, now Pat. No. 11,651,182.

(51) Int. Cl. G06K 19/06; G06F 21/34; G06Q 20/34; G16H 10/65

(52) U.S. Cl. CPC G06K 19/06196; G06K 19/06009; G06F 21/34; G06Q 20/346; G16H 10/65

(57) ABSTRACT

Implementations of a smart card comprising a single smart card for multiple uses which has a number of separate areas that are accessed by one code are provided.

### CROSS REFERENCE TO RELATED APPLICATION

This continuation-in-part application claims the benefit of U.S. patent application Ser. No. 16/527,623, which was filed on Jul. 31, 2019, and is incorporated herein by reference in its entirety.

### TECHNICAL FIELD

This disclosure relates to implementations of a smart card.

### BACKGROUND

People typically have to carry multiple documents and cards from multiple sources. This can be unmanageable.

### BRIEF DESCRIPTION OF THE DRAWINGS

FIG. 1 illustrates an example implementation of a smart card according to the present disclosure.

### DETAILED DESCRIPTION

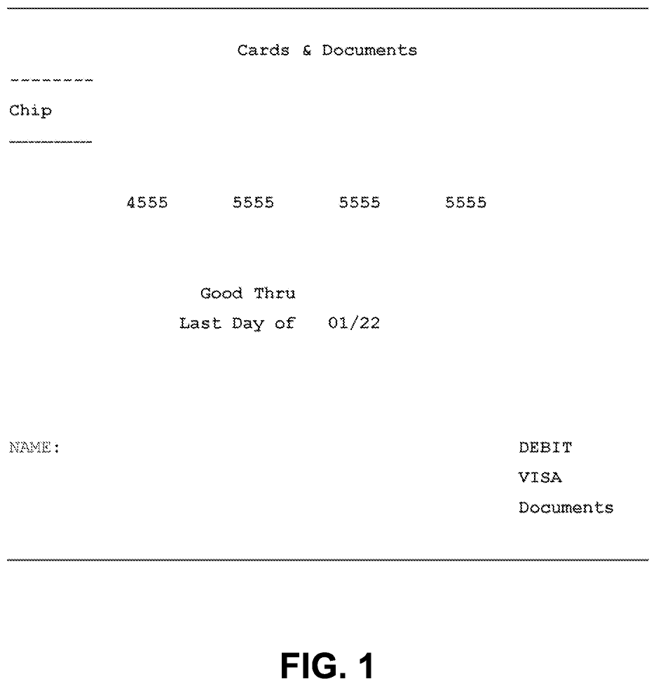

Implementations of cards & documents (a “smart card”) comprising a single smart card for multiple uses which has a number of separate areas that are accessed by one code are provided. As illustrated in FIG. 1, in some implementations, the digital wallet card looks, appears, and is shaped just like a smart card or bank card and provides digital information storage. The card can access several bank cards, credit cards, drivers' license and other personal documentation or identification information. This card eliminates the need to carry multiple transaction cards or documents on one's self like ID card, insurance information, or birth certificate. Instead, all of the person's documents, transaction account information, and documents like medical information are available on one digital wallet card. Authentic copies of documents can be added to a card's chip.

In some implementations, there is provided a smart card made for multiple functions in which it is divided into different sectors (e.g., credit card and bank card transactions and medical insurance). A database of general information, documentation, and identification is combined on one chip card. This all-in-one card system meets maximum security and is easy to use requiring minimum effort to use on a daily basis. A single cipher or pin ensures the safety of the valuable information stored on the card. A customer can make up his/her corresponding secret number due to loss or theft so that no one will be able to receive information from the card except the customer. If the pin is entered incorrectly three times the card will lock automatically. If the customer happens to forget their four-digit code, the customer can, for example, call his/her provider and answer security questions needed to gain access to their card again. In some implementations, this single smart card will work like a phone card and will connect to the provider only which makes it a safe card to have.

In some implementations, with this smart card and card reader, not only can cashless payments be accomplished but healthcare information of a patient's data (e.g., allergy and blood type) can be added on the card's chip. Also, driver's license, birth certificate, social security number and/or any other documentation can be read on the cards chip. With this smart card, carrying around multiple documents and bank cards will cease to exist.

With the digital wallet card solution of the present disclosure, in some implementations, the chip will have the entire person's information and a four-digit code (or multi-digit code) will need to be entered to access the card information. People won't have to carry multiple documents and stacks of cards ever again. Users of this card can add other information, documentation, identification, and bank card information that they desire inside of this digital memory storage smart card. Below is a list of things one card can offer and other documents, information, and documentation can always be added.
1. Drivers license/permit
2. Picture I.D.-Identification
3. Social security card/number
4. Birth certificate
5. Complete Medical History (Illness & Blood Type, etc.)
6. Medical Insurances
7. Medical reports
8. Medical test results
9. Prescription records
10. Life Insurance information
11. Car Insurance information
12. Home Insurance
13. Vehicle registration
14. Credit Card
15. Bank Card
16. Diploma & transcript
17. Educational credentials & Degrees
18. State & federal professional license & Number
19. Credit Scores & History
20. Payment vouchers
21. passports
22. marriage certificates
23. military discharge records
24. fingerprint records
25. membership cards
26. numbers & serial numbers
27. stock shares & bonds
28. any evidence of verifications
29. tax returns
30. deed for land
31. car title
32. financial reports
33. certificates of inspections
34. account statements
35. E-voting over internet
36. Voters I.D.
37. Pilot license
38. Cosmetology license
39. Hazmat & explosives (ATF) license
40. Any other documents can be added later.

In some implementations, to use, access, or enable this card, a security code (e.g., a four-digit code) is used for transaction payments, identification, or documentation. A (POS) transaction machine would be needed for retailers to process transactions due to this new digital wallet. This card will operate just like or similar to bank cards with a swipe or chip in the card to pull up information. If transactions are needed for the smart card online or over a phone, one can enter the numbers on the card just like any other card. Other companion (POS) systems or equipment may be needed for documentation or information access for the card.

This electronic wallet card will combine multiple card accounts and documentation for ease of everyday carrying situations of many cards on one's possession. This all-in-one secure transaction, identity, and documentation card will have capabilities to allow online transactions and access, digital transactions with microprocessor-based smart card solutions. Retailers and other entities will be able to access information documentation, identifications and bank card transaction information stored on the card or associated with the card.

In some implementations, a smart card system of the present disclosure comprises a card having storage embedded therein wherein the storage has stored thereon a plurality of authenticated copies of government issued identity documents, banking information from a plurality of financial accounts, and/or medical information (or any combination of the foregoing) such that the card can be used as a bank card, an identification card, and/or a medical card (or any combination of the foregoing). In some implementations, the smart card system further comprises a card reader configured to retrieve information stored thereon.

In some implementations, a method of using a smart card system of the present disclosure comprises storing one or more government issued identity documents on the card, storing information from one or more financial accounts on the card to conduct financial transactions, and/or storing medical information on the card (or any combination of the forgoing). In some implementations, the method further comprises verifying identity using the card at a card reader in a first transaction requiring identity verification. In some implementations, the method further comprises using the card at a card reader in a second transaction requiring payment to make a payment. In some implementations, the method further comprises verifying medical information using the card at a card reader in a third transaction.

In some implementations, a computer implemented method of using a smart card system of the present disclosure comprises retrieving one or more government issued identity documents from the card and verifying identity using the smart card at a card reader in a first transaction requiring identity verification. In some implementations, the computer implemented method further comprises retrieving medical information from the card and verifying medical information using the card at a card reader in a second transaction.

In some implementations, a computer implemented method of using a smart card system of the present disclosure comprises using a non-government-issued identity card having storage embedded therein, wherein the non-government-issued identity card is not a government-issued identity card. In some implementations, the non-government-issued identity card has an associated number identifying the card wherein the number is printed on the card. In some implementations, the smart card system further comprises a card reader configured to retrieve information stored on the non-government-issued identity card. In some implementations, the smart card system further comprises a storage device accessible over a network.

In some implementations, the computer implemented method comprises storing one or more authenticated copies of government issued identity documents on the storage device.

In some implementations, the computer implemented method comprises storing medical information on the storage device.

In some implementations, the computer implemented method comprises receiving over a network the number identifying the non-government-issued identity card.

In some implementations, the computer implemented method comprises retrieving at least one of the authenticated copies of government issued identity documents or medical information from the storage device using the number identifying the non-government-issued identity card.

In some implementations, the computer implemented method comprises storing one or more authenticated copies of government issued identity documents on the non-government-issued identity card.

In some implementations, the computer implemented method comprises storing authenticated information from one or more financial accounts on the card.

In some implementations, the computer implemented method comprises storing authenticated medical information on the card.

In some implementations, the computer implemented method comprises verifying identity using at least one of the authenticated copies of government issued identity documents stored on the non-government-issued identity card at a card reader in a first transaction requiring identity verification by retrieving the at least one of the authenticated copies of government issued identity documents using the card reader.

In some implementations, the computer implemented method comprises using the card at a card reader in a second transaction requiring payment to make a payment by retrieving information from at least one of the financial accounts stored on the non-government-issued identity card.

In some implementations, the computer implemented method comprises verifying medical information using the authenticated medical information stored on the non-government-issued identity card at a card reader in a third transaction by retrieving authenticated medical information stored on the non-government-issued identity card using the card reader.

In some implementations, manufacturing the card may comprise Ball Grid Array (BGA) and/or Fine Ball Grid Array (FBGA) with embedded passive technologies. The card insides and outer parts may also contain IPX67 and IPX57 for the quality and performance of the card; store machine readers will be able to access card information from the smart card. If the card product happens to need packaging, the packaging will include protection of card information when sending to customers.

The card can come in multiple colors and a four color process along with decorations, pictures, and/or a company's brand mark or logo at the top of the card, which can show quality and ownership.

The figures, including photographs and drawings, comprised herewith may represent one or more implementations of the cards & documents (“smart card”).

Details shown in the figures, such as dimensions, descriptions, etc., are exemplary, and there may be implementations of other suitable details according to the present disclosure.

Reference throughout this specification to “an embodiment” or “implementation” or words of similar import means that a particular described feature, structure, or characteristic is comprised in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations may be depicted in the drawings in a particular order, this should not be understood as requiring that such operations be performed in the particular order shown or in sequential order, or that all illustrated operations be performed, to achieve desirable results.

1. A smart card system comprising:
a card having storage embedded therein wherein the storage has stored thereon at least one authenticated copy of a government issued identity document, banking information from at least one financial account, and medical information such that the card can be used as a bank card, an identification card, and a medical card; and
a card reader configured to retrieve information stored thereon.

2. A method of using the smart card system of claim 1 comprising:
storing at least one government issued identity document on the card;
storing information from at least one of financial account on the card to conduct financial transactions;
storing medical information on the card;
verifying identity using the card at a card reader in a first transaction requiring identity verification;
using the card at a card reader in a second transaction requiring payment to make a payment; and
verifying medical information using the card at a card reader in a third transaction.

3. A computer implemented method of using the smart card system of claim 1, the method comprising,
retrieving at least one government issued identity document from the card and verifying identity using the smart card at a card reader in a first transaction requiring identity verification; and
retrieving medical information from the card and verifying medical information using the card at a card reader in a second transaction.

4. A computer implemented method of using a smart card system comprising a non-government-issued identity card having storage embedded therein, wherein the non-government-issued identity card is not a government-issued identity card, and wherein the non-government-issued identity card has an associated number identifying the card wherein the number is printed on the card, the smart card system further comprising a storage device accessible over a network, the method comprising:
storing at least one authenticated copy of a government issued identity document on the storage device;
receiving over a network the number identifying the non-government-issued identity card; and
retrieving at least one of the authenticated copies of government issued identity documents from the storage device using the number identifying the non-government-issued identity card.

5. The computer implemented method of claim 4, the smart card system further comprising a card reader configured to retrieve information stored on the non-government-issued identity card, the method further comprising:
storing at least one authenticated copy of a government issued identity documents on the non-government-issued identity card; and
verifying identity using at least one of the authenticated copies of government issued identity documents stored on the non-government-issued identity card at a card reader in a first transaction requiring identity verification by retrieving the at least one of the authenticated copies of government issued identity documents using the card reader.

6. The computer implemented method of claim 4, the smart card system further comprising a card reader configured to retrieve information stored on the non-government-issued identity card, the method further comprising storing authenticated information from at least one financial account on the card and using the card at a card reader in a second transaction requiring payment to make a payment by retrieving information from at least one of the financial accounts stored on the non-government-issued identity card.

7. The computer implemented method of claim 4, the smart card system further comprising a card reader configured to retrieve information stored on the non-government-issued identity card, the method further comprising storing authenticated medical information on the card and verifying medical information using the authenticated medical information stored on the non-government-issued identity card at a card reader in a third transaction by retrieving authenticated medical information stored on the non-government-issued identity card using the card reader.

8. The computer implemented method of claim 4 further comprising storing medical information on the storage device and retrieving the medical information from the storage device using the number identifying the non-government-issued identity card.

9. A computer implemented method of using a smart card system comprising a non-government-issued identity card having storage embedded therein, wherein the non-government-issued identity card is not a government-issued identity card, and wherein the non-government-issued identity card has an associated number identifying the card wherein the number is printed on the card, the smart card system further comprising a storage device accessible over a network, the method comprising:
storing medical information on the storage device;
receiving over a network the number identifying the non-government-issued identity card; and
retrieving the medical information from the storage device using the number identifying the non-government-issued identity card.
